SEQUENCE LISTING (10 sequences)

SEQ ID NO: 1 - protein, 280 aa, Krokinobacter eikastus
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWTLYPGAYLMPYLTGVDGFLYSEDGVMARQLVYTIADVSSKVIYGVLLGNLAITLSKNKELVEANS

SEQ ID NO: 2 - protein, 307 aa, Citromicrobium sp. JLT1363
MVAPITDNTVVSPPIPEVNTALANQLGNIENVVTLTMQQYTLGSLILMVSYGACFAFILFFLMSVQNLAPRYRLVPILSAVVMASAGLSLLQEFSLWKDSYAFVDGLYRPLAENETFTNAYRYGNWTITVPILLTQLAIAMGLRQGEIQRRSLRMGVPAVLMIWTGLYGQFGEVGDFSHLNLWGVVSSIFFLWLILEVRQTLIAGISSTPDILKPWPNNLWWFFLATWGLYPIAYALPQLGATAEIVIARQGIYSLADIASKLIYGIILARFVLRRSAYEGYMPAAEALASAPEKPNTGGPGLRRAP

SEQ ID NO: 3 - protein, 303 aa, Citromicrobium bathyomarinum
MAAPIADPTVAEPQIAQADAAISAIENLVTLTSQQYHLGSLILMVSYAAFFAFILFFLMSVQNLAPRYRLVPILSAVVMASAGLTLLQEFGLWKESYAYVDGLYRPLAQNDSFSNAYRYGNWTITVPILLAQLAIAMGLRQAEVHRRALRMAVPGVLMIWTGLYGQFGAVGDFSHLNLWGVISSIFFLWLILEVRQTLIAGLATTPDILKSWPSNLWWFFLASWGLYPLAYALPQLGATGDLVVARQAIYSFADIASKLIYGIILARFVLRRSAFEGYMPAAEALAPTPDKPNTGGPGLRRAP

SEQ ID NO: 4 - protein, 296 aa, Fulvimarina pelagi
MQDTELFADQLGNIENLVSLTVGQYTMGSLILMVSYGAHFAFVLYFLMTSLQLAPRYRIVPIMSAIVMVSAGLSLLREFNAWEQSYEFVEGMYQPLAENSTFTNAYRYGNWTITVPILLTQLPLAFGLLRPELHVRAARMCIPALLMIWTGLVGQFGETGNYLRLNVWGVISTIFFVWLIIEVRGVISRAIAMGPAELAAWPKNIWWFFLAFWGLYPIAYALPQLGHTGDIVVIRQLLYSIADVFSKLVYGIILSRYVLRRSALEGYKPAIEALSRTPVDLSMSKNGGAGYGDGDR

SEQ ID NO: 5 - protein, 284 aa, Chaetoceros neogracile
MSAVLNVEGVRAACDRETGAAIANMENCLQYSPLQFELVGHALVIGYAAQAAGFIYFAMTMNMTKGRNYQLCSIYGMIVMLSAFLLLYNQWAAWEDSFVLNAAGLYESGGVKLFSNGYRYLNWSIDVPLLLLQLVLVSGLEVGTGFNKNLQVTTSGLLMIYLGYIGQFYENPDSMVPLIAFGVVGTVAYAIMLAIVLQCLSHAKKNFKTESAKFKMNLVFWIFLIFWTIYPISYFMPVFSYTAEGVVVRQFIYTIADVVSKVIYCIILTQICMTTVMLLREQIA

SEQ ID NO: 6 - protein, 294 aa, Truepera radiovictrix
MLTMENLLTYSPVSYSIVSNTLTLGYAAMAAGLVYFVTTSKRAAPPYRLSSTLSAVVMVSAFLALYQLHQTWLSAFTFNGEVWEGGATAFNNGYRYINWSIDVPILLTQLLIVMGFTGARFRRLWLQFVVAGLAMIYTGYAGQFYEATDSARLYLWGAISTAFFLWILVLVRRTIFDPPDALPERAAGLMRGVWWVLLGSWLLYPGAYLMPVFALSEGGVVARQISFTVADVVSKVIYGVMLSRVAELRSQADGYAHALESETQPNPDRRGGAGVAAVQPQGGRAPRKGRVHRS

SEQ ID NO: 7 - protein, 294 aa, T. radiovictrix DSM 17093
MQPSPHRLGNATFENYVGLESGYTEMAYQMSAHVLTVGYAIMLAALIYFILTIRQVRPRYRMSSILSVVVMVSAFLLLLIQQLNWTSALQFDPATARYRLAPEGVEGIVTAGDLFNNGYRYLNWLIDVPMLLFQILFVVTLSRSSFASVRNQFWFSGVAMIITGYVGQFYEVTRPGLFFLWGSISTVFFIHILIVMRRVIKEGVENAPDSAKGMLGAIWPLFLISWMLYPGAYLMPYLYSFSLEPALSESAVVARHLTYTVADVTSKVIYGVLLAAAATRMSKAEGYDWEVQTA

SEQ ID NO: 8 - protein, 341 aa, Gillisia limnaea
MFGDFNRVDNPILSLSYFKIQLTGIGLCRLLKPCLNFSKNIKHIKKFIKINGNKFEIDYFDMTQELGNANFENFIGATEGFSEIAYQFTSHILTLGYAVMLAGLLYFILTIKKVDKKYQMSNILSAVVMVSAFLLLYAQAENWTTSFTFDISRGKYFLEPNGDLFNNGYRYLNWLIDVPMLLFQILFVVSLTKSKFSSIRNQFWFSGAMMIITGYIGQFYEVSNLTAFFVWGAISSVFFFHILWVMKKVINEGKEGLSADAQKILSNIWVLFLVSWFLYPGAYLMPYLTGLDGFFFSEDGVMARQLTYTIADVCSKVIYGVLLGNLALKLSNNKEMVELSN

SEQ ID NO: 9 - protein, 280 aa, Krokinobacter (Dokdonia) sp. 4H-3-7-5
MTQELGNANFENFIGATEGFSEIAYQFTSHILTLGYAVMLAGLLYFILTIKNVDKKFQMSNILSAVVMVSAFLLLYAQAQNWTSSFTFNEEVGRYFLDPSGDLFNNGYRYLNWLIDVPMLLFQILFVVSLTTSKFSSVRNQFWFSGAMMIITGYIGQFYEVSNLTAFLVWGAISSAFFFHILWVMKKVINEGKEGISPAGQKILSNIWILFLISWFLYPGAYLMPYLTGVDGFLYSEDGVMARQLVYTIADVCSKVIYGVLLGNLAITLSKNKELVEANS

SEQ ID NO: 10 - protein, 8 aa, Artificial Sequence
  C-terminal tag
LEHHHHHH

(19) United States
(12) Patent Application Publication
(10) Pub. No.: US 2017/0362281 A1
(43) Pub. Date: Dec. 21, 2017

(54) MUTANT NQ-RHODOPSIN KR 2

(71) Applicants: MAX-PLANCK-GESELLSCHAFT ZUR FOERDERUNG DER WISSENSCHAFTEN E.V., Muenchen (DE); FORSCHUNGSZENTRUM JUELICH GMBH, Jülich (DE); CEA COMMISSARIAT À L'ÉNERGIE ATOMIQUE ET AUX ÉNERGIES ALTERNATIVES, Fontenay-aux-Roses Cedex (FR)

(72) Inventors: Ernst BAMBERG, Kelkheim (DE); Christian BAMANN, Frankfurt (DE); Vitaly POLOVINKIN, Grenoble (FR); Valentin GORDELIY, Laffrey (FR); Vitaly SHEVCHENKO, Juelich (DE); Ivan GUSHCHIN, Juelich (DE)

(73) Assignees: MAX-PLANCK-GESELLSCHAFT ZUR FOERDERUNG DER WISSENSCHAFTEN E.V., Muenchen (DE); FORSCHUNGSZENTRUM JUELICH GMBH, Jülich (DE); CEA COMMISSARIAT À L'ÉNERGIE ATOMIQUE ET AUX ÉNERGIES ALTERNATIVES, Fontenay-aux-Roses Cedex (FR)

(21) Appl. No.: 15/538,996

(22) PCT Filed: Dec. 23, 2015
(86) PCT No.: PCT/EP2015/081177

§ 371 Date: Jun. 22, 2017

(30) Foreign Application Priority Data
Dec. 23, 2014 (EP) 14200190.8

(51) Int. Cl. C07K 14/195; A61K 38/16; G01N 33/50; C12N 5/0793; A61K 48/00

(52) U.S. Cl. CPC C07K 14/195; G01N 33/5041; C12N 5/0619; G01N 33/5058; A61K 38/164; A61K 48/00; C12N 2510/00; C12N 2501/998

(57) ABSTRACT

The invention relates to mutant NQ-Rhodopsin having potassium pumping properties, nucleic acid constructs encoding same, expression vectors carrying the nucleic acid construct, cells comprising said nucleic acid construct or expression vector, and their respective uses.

The invention relates to mutant NQ-Rhodopsin having potassium pumping properties, nucleic acid constructs encoding same, expression vectors carrying the nucleic acid construct, cells comprising said nucleic acid construct or expression vector, and their respective uses.

### BACKGROUND OF THE INVENTION

Precise maintenance of the right gradients of ions such as H⁺, K⁺, Na⁺and Cl⁻, across the cellular membranes is a principal feature of all living organisms. In some bacteria, archaea and unicellular eukaryota these gradients are created by proteins of the microbial rhodopsins family. While the light-driven proton- and anion-pumping rhodopsins have been discovered long ago (Oesterhelt, D. & Stoeckenius, Nature 233, 149-152 (1971). Schobert, B. & Lanyi, J. K. J. Biol. Chem. 257, 10306-10313 (1982)), the light-driven cation pumps until recently have not been known, and even their existence has been questioned. Recently, a novel class of microbial rhodopsins, dubbed NQ-rhodopsins, was found in marine flavobacteria (Kwon, S.-K. et al. Genome Biol Evol 5, 187-199 (2013); KR20130134792; KR20140011750). In the NQ-rhodopsins, the typical proton acceptor of proton pumps, aspartate, is replaced by asparagine (N), and the typical proton donor, glutamate, is replaced by glutamine (Q). Besides that, there is a unique aspartate amino acid (residue 116 in KR2) in the retinal vicinity.

One member of the NQ-rhodopsins, Krokinobacter eikastus rhodopsin 2 (KR2), was shown to be a selective light-driven sodium pump (Inoue, K. et al. Nat Commun 4, 1678 (2013)). The light-driven sodium pump KR2 is composed of seven transmembrane helices, A to G, connected by three intracellular and three extracellular loops, and a short N-terminal α-helix capping the inside of the protein (FIG. 1). This helix is unique among the microbial rhodopsins of known structure. The B-C loop is elongated and forms a β-hairpin that is also involved in β-sheet interactions with the linker between the helix A and the N-terminal α-helix. Overall, the structure of the KR2 transmembrane part displays features similar to those of other microbial rhodopsins (FIG. 2).

Santacruz-Toloza et al. Biochemistry, 33(6), 1295-1299 (1994) disclose a system in which voltage-dependent D. melanogaster ‘Shaker’ potassium channels have been assayed in reconstituted vesicles using a voltage-control system driven by bacteriorhodopsin, a light activated proton pump.

In the fast expanding field of optogenetics (see, for example, Boyden, et al. Nat. Neurosci. 8, 1263-1268 (2005)), there is an ongoing need for engineering light-driven proteins with valuable novel properties.

### SUMMARY OF THE INVENTION

Maintenance of the membrane electrochemical potential and ion gradient between the interior and exterior of biological cells or organelles is a key and common process present in the organisms from all domains of life. The gradient is supported by various active transporters, among which are light-driven pumps of the microbial rhodopsin family of proteins. Until the recent discovery of the sodium-pumping rhodopsins (Kwon, S.-K. et al. Genome Biol Evol 5, 187-199 (2013); Inoue, K. et al. Nat Commun 4, 1678 (2013)), only the proton and anion pumps were known (Ernst, O. P. et al. Chem. Rev. 114, 126-163 (2014); Grote, M., Engelhard, M. & Hegemann, P. Biochimica et Biophysica Acta (BBA)-Bioenergetics 1837, 533-545 (2014)).

Consequently, the molecular mechanism of the sodium translocation was also not known. The inventors were able to solve the structures of the light-driven sodium pump KR2 from Krokinobacter eikastus in several crystal forms at the resolutions of 1.45, 2.2 and 2.8 Å, in the monomeric blue and pentameric red states. The structures have revealed in detail the sodium/proton translocation pathway (cf. FIG. 3). The ion uptake cavity, unexpectedly protruding from the surface towards Gln-123, appears to be the selectivity filter of the pump. Obstruction of the cavity with mutation G263F imparts KR2 with the ability to pump potassium. This understanding allowed the inventors to develop a potassium-pumping mutant of KR2.

Accordingly, the present disclosure relates to a light-driven cation pump (cation channel), wherein the light-driven cation pump comprises an amino acid sequence which has at least 70% sequence identity to the full length amino acid sequence of SEQ ID NO: 1 (Krokinobacter eikastus KR2), and which comprises a substitution at a position corresponding to G263 in SEQ ID NO: 1, which substitution confers a potassium ion pumping ability.

Further disclosed is a nucleic acid construct, comprising a nucleotide sequence encoding said light-driven cation pump; an expression vector, comprising said nucleotide sequence coding for the light-driven cation pump or said nucleic acid construct; and a recombinant host cell comprising said light-driven cation pump, said nucleic acid construct or said expression vector; as well as therapeutic and non-therapeutic uses thereof.

Finally, a non-therapeutic method, in particular an in vitro method, of actively transporting potassium ions across a membrane, preferably a biologic membrane, is provided, comprising the step of introducing said light-driven cation pump into said membrane.

### DETAILED DESCRIPTION OF THE PREFERRED EMBODIMENTS

The present disclosure provides a light-driven cation pump, wherein the light-driven cation pump comprises an amino acid sequence which has at least 70% sequence identity to the full length amino acid sequence of SEQ ID NO: 1 (Krokinobacter eikastus KR2), and which comprises a substitution at a position corresponding to G263 in SEQ ID NO: 1, which substitution confers a potassium ion pumping ability.

Wild-type Krokinobacter eikastus KR2 has the following amino acid sequence:

MTQELGNANFENFIGATEGFSETAYQFTSHILTLGYAVMLAGLLYFILTI
KNVDKKFQMSNILSAVVMVSAFLLLYAQAQNWTSSFTFNEEVGRYFLDPS
GDLFNNGYRYLNWLIDVPMLLFQILFVVSLTTSKFSSVRNQFWFSGAMMI
ITGYIGQFYEVSNLTAFLVWGAISSAFFFHILWVMKKVINEGKEGISPAG
QKILSNIWILFLISWTLYPGAYLMPYLTGVDGFLYSEDGVMARQLVYTIA
DVSSKVIYGVLLGNLAITLSKNKELVEANS

In a preferred embodiment, the light-driven cation pump comprises an amino acid sequence which has at least 70% identity, preferably at least 75% identity, more preferably at least 80% identity, more preferably at least 85% identity, more preferably at least 88% identiy, more preferably at least 90% identity, more preferably at least 91% identity, more preferably at least 92% identity, more preferably at least 93% identity, more preferably at least 94% identity, more preferably at least 95% identity, more preferably at least 96% identity, more preferably at least 97% identity, more preferably at least 98% identity, and even more preferably at least 99% identity over the full length of the amino acid sequence shown in SEQ ID NO: 1. In a most preferred embodiment, the light-driven cation pump comprises, the amino acid sequence of SEQ ID NO: 1 (KR2), except for a substitution at position G263 in SEQ ID NO: 1, which substitution confers a potassium ion pumping ability. For example, the light driven cation pump may further comprise an N- terminal or C-terminal extension, a small linker peptide of up to 20-25 residues; or a small extension that facilitates purification by changing net charge or another function, such as an affinity tag, an antigenic epitope or a binding domain. Preferably the extension is an affinity tag, more preferably a C-terminal affinity tag. In particular, the affinity tag may be a polyhistidine tract, most preferably the tag LEHHHHHH (SEQ ID NO: 10).

In a still most preferred embodiment, the light-driven cation pump consists of the amino acid sequence of SEQ ID NO: 1 (KR2), except for a substitution at position G263 in SEQ ID NO: 1, which substitution confers a potassium ion pumping ability.

Preferably, the light-driven cation pump comprises a further substitution at a position corresponding to S254 in SEQ ID NO: 1, since the amino acid Ser in the position corresponding to position 254 of SEQ ID NO. 1 is highly conserved among identified sodium pumping rhodopsins. More preferably, said substitution is S254A.

Generally, an amino acid sequence has “at least x % identity” with another amino acid sequence or SEQ ID NO: 1 above, when the sequence identity between those to aligned sequences is at least x % over the full length of SEQ ID NO: 1. Such an alignment can be performed using for example publicly available computer homology programs such as the “BLAST” program provided at the NCBI homepage at http://www.ncbi.nlm.nih.gov/blast/blast.cgi, using the default settings provided therein. Further methods of calculating sequence identity percentages of sets of nucleic acid sequences are known in the art.

Examples for such light-inducible ion channels comprising an amino acid sequence which has at least 70% identity to the amino acid sequence of SEQ ID NO: 1 are shown in FIG. 6.

The substitution at position G263 in SEQ ID NO: 1, or at the position corresponding to G263 in SEQ ID NO: 1 may be any substitution, which confers a potassium ion pumping ability. The term “potassium ion pumping ability” as used herein may be tested as follows: The mutant light-driven cation pump in question is reconstituted into liposomes (80% POPC, 10% POPG, 10% cholesterol (w/w), Avanti/Sigma Aldrich) at protein/lipid ratio of 7:100 (w/w). The detergent is removed by overnight stirring at 4° C. with the detergent-absorbing beads (Bio-Beads SM 2, BioRad). Optically BLMs are formed as described previously (Bamberg, E. et al. Biophys. Struct. Mechanism 5, 277-292 (1979), which is incorporated herein by reference). The electrolyte solution is 20 mM HEPES pH 7.4 without any Na⁺ or K⁺ ions. Mutant light-driven cation pump-containing proteoliposomes are added to one of the compartments under gentle stirring. The system is illuminated with a mercury arc lamp (Osram HBO 100) at wavelengths >455 nm. Photosensivity of the samples developes over time and reaches maximal current amplitudes after ˜30 minutes. 2 μM of the protonophore 1799 ((2,6-dihydroxy)-1,1,1,7,7,7-hexafluoro-2,6-bis(trifluoromethyl)-heptane-4-one) is added to both compartments, which effectively permeabilizes the compound membrane system for protons. Potassium titration is performed with KCl solutions by addition to both compartments of the cuvette. The system conductance remained constant during the titration. Subsequently, the membrane is, made permeable for the cations, too, by the addition of the exchanger monensin. Photocurrents are measured under short-circuit conditions, so that no external driving force is generated. Further details of the system are described in Bamberg, E. et al. Biophys. Struct. Mechanism 5, 277-292 (1979), incorporated herein by reference. A drastic increase of the photocurrents upon addition of potassium ions is indicative of potassium ion pumping ability. The turnover of potassium ions can be calculated from the photocurrents. Preferably, the mutant light-driven cation pump has a turnover for potassium ions of more than 10/s, more preferably more than 20/s, more preferably more than 30/s, even more preferably more than 40/s and most preferably more than 50/s. In a most preferred embodiment, the mutant light-driven cation pump has a turnover for potassium ions of 50-70/s. The slowest step in the photocycle is 1-100 ms, more preferably 5-75 ms, and most preferably 10-50 ms. In a very preferred embodiment, the mutation is selected from G263F and G263Y, more preferably the substitution is G263F.

In addition, the light-driven cation pump comprises further (semi-)conservative substitutions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Typical semi-conservative and conservative substitutions are:

| | Conservative | |
|---|---|---|
| Amino acid | substitution | Semi-conservative substitution |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Furthermore, the skilled person will appreciate that glycines at sterically demanding positions should not be substituted and that proline should not be introduced into parts of the protein which have an alpha-helical or a beta-sheet structure. In still one preferred embodiment, the light-driven cation pump does not comprise any substitution at a position in which the corresponding amino acid residue is identical among all NQ-rhodopsins in the alignment of FIG. 6. On the other hand, substitutions of SEQ ID NO: 1 by amino acid residues which are shown to correspond to the amino acid sequence of SEQ ID NO: 1 (said positions are indicated by an underlinement in SEQ ID NO: 1 in FIG. 6) are contemplated. In still another preferred embodiment, the mutant light-driven cation pump disclosed herein is a non-naturally occurring cation pump, i.e. a potassium ion pump derived by genetic engineering from a parent cation pump, which has no phenylalanine at position 263, when aligned with SEQ ID NO: 1.

In general, the retinal or retinal derivative necessary for the functioning of the light-driven cation pump is produced by the cell to be transfected with said channel. Depending on its conformation, the retinal may be all-trans retinal, 11-cis-retinal, 13-cis-retinal, or 9-cis-retinal. However, it is also contemplated that the mutant light-driven cation pump of the invention may be incorporated into vesicles, liposomes or other artificial cell membranes. Accordingly, in a particular embodiment, the mutant light-driven cation pump as described above, further comprises, in non-covalent linkage, a retinal or retinal derivative, preferably wherein the retinal derivative is selected from the group consisting of 3,4-dehydroretinal, 13-ethylretinal, 9-dm-retinal, 3-hydroxyretinal, 4-hydroxyretinal, naphthylretinal; 3,7,11-trimethyl-dodeca-2,4,6,8,10-pentaenal; 3,7-dimethyl-deca-2,4,6,8-tetraenal; 3,7-dimethyl-octa-2,4,6-trienal; and 6-7 rotation-blocked retinals, 8-9 rotation-blocked retinals, and 10-11 rotation-blocked retinals.

The present disclosure further relates to a nucleic acid construct, comprising a nucleotide sequence encoding the mutant light-driven cation pump as disclosed herein. To ensure optimal expression, the coding DNA can also be suitably modified, for example by adding suitable regulatory sequences and/or targeting sequences and/or by matching of the coding DNA sequence to the preferred codon usage of the chosen host. The targeting sequence may encode a C-terminal extension targeting the mutant light-driven cation pump to a particular site or compartment within the cell, such as to the synapse or to a post-synaptic site, to the axon-hillock, or the endoplasmic reticulum. The nucleic acid may be combined with further elements, e.g., a promoter and a transcription start and stop signal and a translation start and stop signal and a polyadenylation signal in order to provide for expression of the sequence of the protein of the invention. The promoter can be inducible or constitutive, general or cell specific promoter. An example of a cell-specific promoter is the mGlu6-promotor specific for bipolar cells. Selection of promoters, vectors and other elements is a matter of routine design within the level of ordinary skill in the art. Many such elements are described in the literature and are available through commercial suppliers.

Further disclosed is an expression vector, comprising a nucleotide sequence coding for the mutant light-driven cation pump disclosed herein or the nucleic acid construct as disclosed herein. In a preferred embodiment, the vector is suitable for gene therapy, in particular wherein the vector is suitable for virus-mediated gene transfer. The term “suitable for virus-mediated gene transfer” means herein that said vector can be packed in a virus and thus be delivered to the site or the cells of interest. Examples of viruses suitable for gene therapy are retroviruses, adenoviruses, adeno-associated viruses, lentiviruses, pox viruses, alphaviruses, rabies virus, semliki forest virus and herpes viruses. These viruses differ in how well they transfer genes to the cells they recognize and are able to infect, and whether they alter the cell's DNA permanently or temporarily. However, gene therapy also encompasses non-viral methods, such as application of naked DNA, lipoplexes and polyplexes, and dendrimers.

As described above, the resulting nucleic acid sequence may be introduced into cells e.g. using a virus as a carrier or by transfection including e.g. by chemical transfectants (such as Lipofectamine, Fugene, etc.), electroporation, calcium phosphate co-precipitation and direct diffusion of DNA. Transfection with DNA yields stable cells or cell lines, if the transfected DNA is integrated into the genome, or unstable (transient) cells or cell lines, wherein the transfected DNA exists in an extrachromosomal form. Furthermore, stable cell lines can be obtained by using episomal replicating plasmids, which means that the inheritance of the extrachromosomal plasmid is controlled by control elements that are integrated into the cell genome. In general, the selection of a suitable vector or plasmid depends on the intended host cell.

Accordingly, further disclosed is a recombinant host cell comprising the mutant light-driven cation pump, the nucleic acid construct, or the expression vector of the present disclosure. Preferably, the host cell does not comprise a naturally occurring light-driven potassium pump as disclosed herein. As noted above, the incorporation of the mutant light-driven cation pump into the membrane of cells which do not express the corresponding channels in nature can for example be simply effected in that, using known procedures of recombinant DNA technology, the DNA coding for this ion channel is firstly incorporated into a suitable expression vector, e.g. a plasmid, a cosmid or a virus, the target cells are then transformed with this, and the protein is expressed in this host. Next, the cells are treated in a suitable manner, e.g. with retinal, in order to enable the linkage of a Schiffs base between protein and retinal. As noted above, this is not necessary for many cells, in particular for cells in vivo, since the retinal or retinal derivative is produced by the cell to be transfected with said channel. For example, this may be carried out in various yeasts such as Saccharomyces cerevisiae, Schizosaccharomyces pombe or Pichia pastoris as already successfully performed for rhodopsins such as bacteriorhodopsin and/or bovine rhodopsin. Alternatively, the mutant light-driven cation pump may be expressed in Xenopus oocytes, such as Xenopus laevis oocytes, which represent commonly used cell systems in research. The expression can also be effected in certain mammalian cell systems or insect cell systems. The expression may be effected either with episomal vectors as transient expression, preferably in COS cells (generated by infection of “African green monkey kidney CV1” cells) or HEK cells (“human embryonic kidney cells”, e.g. HEK293 cells), or BHK-cells (“baby hamster kidney cells”), or in the form of stable expression (by integration into the genome) in CHO cells (“Chinese hamster ovary cells”), or MDCK cells (“Madine-Darby canine kidney cells”). However, preferably the cell is a mammalian cell.

Still more preferably, the cell is an electrically excitable cell. In an even more preferred embodiment, the mammalian cell is a neuron. A neuron is an electrically excitable cell that processes and transmits information by electrical and chemical signalling, wherein chemical signaling occurs via synapses, specialized connections with other cells. A number of specialized types of neurons exist such as sensory neurons responding to touch, sound, light and numerous other stimuli affecting cells of the sensory organs, motor neurons receiving signals from the brain and spinal cord and causing muscle contractions and affecting glands, and interneurons connecting neurons to other neurons within the same region of the brain or spinal cord. Generally, a neuron possesses a soma, dendrites, and an axon. Dendrites are filaments that arise from the cell body, often extending for hundreds of microns and branching multiple times. An axon is a special cellular filament that arises from the cell body at a site called the axon hillock. The cell body of a neuron frequently gives rise to multiple dendrites, but never to more than one axon, although the axon may branch hundreds of times before it terminates. At the majority of synapses, signals are sent from the axon of one neuron to a dendrite of another. There are, however, many exceptions to these rules: neurons that lack dendrites, neurons that have no axon, synapses that connect an axon to another axon or a dendrite to another dendrite, etc. Most neurons can further be anatomically characterized as unipolar or pseudounipolar (dendrite and axon emerge from same process), bipolar (axon and single dendrite on opposite ends of the soma), multipolar (having more than two dendrites and may be further classified as (i) Golgi I neurons with long-projecting axonal processes, such as pyramidal cells, Purkinje cells, and anterior horn cells, and (ii) Golgi II: neurons whose axonal process projects locally, e.g., granule cells. Accordingly, the neuron may preferably be a bipolar neuron, a pseudounipolar neuron, a multipolar neuron, a an anaxonic neuron, a basket cell, Betz cell, Lugaro cell, medium spiny neuron, Purkinje cell, pyramidal neuron, Renshaw cell, unipolar brush cell, granule cell, anterior horn cell, spindle cell, an afferent neuron, efferent neuron, or an interneuron.

Neurons are responsible for conveying commands to the muscles, and move muscles in response to voluntary and involuntary (automatic) signals. In addition, muscles react to reflexive nerve stimuli. Mammalian nerves that control skeletal muscles correspond with neuron groups along the primary motor cortex of the cerebral cortex of the brain. Commands are routed through the basal ganglia and modified by input from the cerebellum before being routed through the pyramidal tract to the spinal cord and the motor end plate at the muscles. Feedback, e.g., of the extrapyramidal system contribute signals to influence muscle tone and response. In the muscles, in turn, the muscle spindles convey information about the degree of muscle length and stretch to the central nervous system to assist in maintaining posture and joint position. Thus, in another still more preferred embodiment, the mammalian cell is a muscle cell. Muscle cells contain filaments of actin and myosin that slide past one another, which changes both the shape and length of the cell, and ultimately results in contraction. In a preferred embodiment, the muscle cell is a skeletal muscle cell, a smooth muscle cell, or a cardiac muscle cell.

For EPO proceedings, and/or depending on the patentability requirements in the respective jurisdictions, the cell may not be a human embryonic stem cell, produced using a process which involves modifying the germ line genetic identity of human beings or which involves use of a human embryo for industrial or commercial purposes.

The cell may be isolated (and genetically modified), maintained and cultured at an appropriate temperature and gas mixture (typically, 37° C., 5% CO₂), optionally in a cell incubator as known to the skilled person and as exemplified for certain strains, cell lines, or cell types in the examples. Culture conditions may vary for each cell type, and variation of conditions for a particular cell type can result in different phenotypes. Aside from temperature and gas mixture, the most commonly varied factor in cell culture systems is the growth medium. Recipes for growth media can vary in pH, glucose concentration, growth factor and the presence of other nutrient components among others. Growth media are either commercially available, or can be prepared according to compositions, which are obtainable from the American Tissue Culture Collection (ATCC). Growth factors used for supplement media are often derived from animal blood such as calf serum.

Additionally, antibiotics may be added to the growth media. Amongst the common manipulations carried out on culture cells are media changes and passaging cells.

The mutant light-driven cation pump, the nucleic acid construct or the expression vector as disclosed herein encoding same, or the recombinant host cell disclosed herein expressing same are contemplated to be highly useful as a research tool, preferably as an optogenetic research tool. A cell's membrane potential is due to the intracellular high concentration of potassium ion, and low concentration of sodium ions. The presently disclosed potassium pumping mutant cation pump is capable of pumping potassium ions outwards of the cell, thereby silencing an electrically excitable cell. Ion pumping rhodopsins have been previously used as neural silencers (cf., Zhang et al. Nature. 446(7136), 633-639 (2007); Deisseroth, K. Nat. Methods 8, 26-29 (2011); and Chow et al. Nature 463, 98-102 (2010); all incorporated herein by reference), and it is contemplated that the presently disclosed mutant light-driven cation pump will be likewise useful. Accordingly, the mutant light-driven cation pump, the nucleic acid construct, and/or the expression vector as disclosed herein may be used in vitro as a silencer of electrically excitable cells. Further contemplated is a non-therapeutic method of actively transporting potassium ions across a membrane, preferably a biologic membrane, comprising the step of introducing the light-driven cation pump of the present disclosure into said membrane. In a more preferred embodiment, the method is an in vitro method.

On the other hand, the mutant light-driven cation pump, the nucleic acid construct, and/or the expression vector as disclosed herein may be used in vivo as a medicament. For example, the mutant light-driven cation pump, the nucleic acid construct, and/or the expression vector as disclosed herein may be used as a silencer of electrically excitable cells, such as an anaesthetic or as a relaxant.

Finally, the present disclosure also contemplates the use of the mutant light-driven cation pump, the nucleic acid construct, the expression vector, or the recombinant host cell of the present disclosure in a method of high-throughput screening. A high-throughput screening (HTS), is a method for scientific experimentation especially used in drug discovery and relevant to the fields of biology and chemistry. HTS allows a researcher to effectively conduct millions of biochemical, genetic or pharmacological tests in a short period of time, often through a combination of modern robotics, data processing and control software, liquid handling devices, and sensitive detectors. By this process, one may rapidly identify active agents which modulate a particular biomolecular pathway; particularly a substance modifying an ion channel, such as the light-driven cation pump according to the present disclosure, a Ca⁺⁺-inducible potassium channel, or a BK channel. Thus, upon excitation, one will receive a change in the membrane potential, which may be monitored by potential-sensitive dyes such as RH 421 (N-(4-Sulfobutyl)-4-(4-(4-(dipentylamino)phenyl)butadienyl)pyridinium, inner salt). In essence, HTS uses an approach to collect a large amount of experimental data on the effect of a multitude of substances on a particular target in a relatively short time. A screen, in this context, is the larger experiment, with a single goal (usually testing a scientific hypothesis), to which all this data may subsequently be applied. For HTS, cells according to the present disclosure may be seed in a tissue plate, such as a multi well plate, e.g. a 96-well plate. Then the cell in the plate is contacted with the test substance for a time sufficient to interact with the targeted ion channel. The test substance may be different from well to well across the plate. After incubation time has passed, measurements are taken across all the plate's wells, either manually or by a machine and optionally compared to measurements of a cell which has not been contacted with the test substance. Manual measurements may be necessary when the researcher is using patch-clamp, looking for effects not yet implemented in automated routines. Otherwise, a specialized automated analysis machine can run a number of experiments on the wells (such as analysing light of a particular frequency or a high-throughput patch-clamp measurement). In this case, the machine outputs the result of each experiment e.g. as a grid of numeric values, with each number mapping to the value obtained from a single well. Depending upon the results of this first assay, the researcher can perform follow up assays within the same screen by using substances similar to those identified as active (i.e. modifying an intracellular cyclic nucleotide level) into new assay plates, and then re-running the experiment to collect further data, optimize the structure of the chemical agent to improve the effect of the agent on the cell. Automation is an important element in HTS's usefulness. A specialized robot is often responsible for much of the process over the lifetime of a single assay plate, from creation through final analysis. An HTS robot can usually prepare and analyze many plates simultaneously, further speeding the data-collection process. Examples for apparatuses suitable for HTS in accordance with the present invention comprise a Fluorometric Imaging Plate Reader (FLIPR™; Molecular Devices), FLEXstation™ (Molecular Devices), Voltage Ion Probe Reader (VIPR, Aurora Biosciences), Attofluor® Ratio Vision® (ATTO).

In the following, the present invention is illustrated by figures and examples which are not intended to limit the scope of the present invention.

### DESCRIPTION OF THE FIGURES

FIG. 1A, Overall architecture of KR2. KR2 fold and its orientation in the membrane. B. Tested mutations in the KR2 ion pathway. The mutants N61M, and G263F,L are described herein, the mutants R109A, N112A,D, D116A,E,N, Q123A,E and U2b1A,E,N have been tested by Inoue et al. (Nat Commun 4, 1678 (2013)).

FIG. 2 Comparison of KR2 structure with structures of other microbial rhodopsins. a, Comparison of the KR2 backbone structure in the type A crystals (yellow) with those of bacteriorhodopsin (BR, purple, PDB ID 1C3W) and halorhodopsin (HR, blue, PDB ID 1E12). Root mean square deviations of the KR2 transmembrane helix backbone N, C and C_{α} atoms relative to BR and HR are 2.5 and 2.3 Å correspondingly. b Comparison of the cytoplasmic sides. c, Comparison of the retinal positions. In KR2, the retinal is closer to the helices C and D, similar to blue proteorhodopsins (grey, PDB ID 4JQ6, salmon, PDB ID 4KLY) and opposite to BR, HR and Exiguobacterium sibiricum rhodopsin (green, PDB ID 4HYJ). d, Function-defining residues in KR2, BR and HR. While BR's Asp-112 is present in all the three proteins, the residues Asp-85/Thr-89/Asp-96 are replaced with Asn-112/Asp-116/Gln-123 in KR2 and other sodium pumps, earning them the designation NQ- or NDQ-rhodopsins.

FIG. 3. Ion translocation pathway in KR2. a, Putative ion translocation pathway residues inside KR2. N-terminal helix is shown in blue, helix A is shown in transparent pink. b, Water molecules (red) and cavities (transparent red) inside KR2. The black arrows show the putative ion path. Helices F and G are not shown. The cavities were calculated using HOLLOW (Ho, B. & Gruswitz, F. BMC Structural Biology 8, 49 (2008)). In this illustration, structure of the monomeric blue KR2 was used.

FIG. 4. Ion uptake cavity in KR2. a, The ion uptake cavity (red). The protrusion is absent in other microbial rhodopsins due to bulky amino acids at KR2′s G263 position, such as Leu-224 in bacteriorhodopsin (Luecke, H., et al. Journal of Molecular Biology 291, 899-911 (1999)) (BR), Leu-250 in halorhodopsin (Kolbe, M., et al. Science 288, 1390-1396 (2000)) (HR) and Phe-233 in Exiguobacterium sibiricum rhodopsin (Gushchin, I. et al. PNAS 110, 12631-12636 (2013)). (ESR). b, Effects of the N61M, G263F and G263L mutations on the activity of the protein in E. coli cells. Addition of the protonophore CCCP allows the protons to enter the cell in exchange for the pumped ions, enhancing the alkalization of the media. c, Comparison of the wild type and G263F mutant photocurrents in the liposomes attached to BLMs in presence of the protonophore 1799. Illumination starts at 0.5 s and stops at 16.5 s (NaCl measurements) or 4.5 s (KCl measurements).

FIG. 5 Pump activities of KR2 mutants expressed in E. coli. The solutions contain 100 mM NaCl (blue), 100 mM NaCl and 30 μM CCCP (green), 100 mM KCl (red), 100 mM KCl and 30 μM CCCP (magenta). The cells were illuminated for 300 s (light area on the plots). From highest to lowest, the plots show: WT (green->blue->red); WT pH 5.6 (blue->green->red); N61M (green->blue->red); G263F (green->magenta->blue->red); G263L (green->blue/magenta/red).

FIG. 6 Sequence alignment using the alignment program COBALT (publicly available at http://www.st-va.ncbi.nlm.nih.gov/) and default settings. The aligned sequences (accession numbers in brackets) are: 1: Krokinobacter eikastus KR2 (Uniprot ID N0DKS8; SEQ ID NO: 1) as used in the examples as the parent enzyme; 2: Citromicrobium sp. JLT1363 (ZP_08702831; SEQ ID NO: 2); 3: Citromicrobium bathyomarinum JL354 (ZP_06860850; SEQ ID NO: 3); 4: Fulvimarina pelagi HTCC2506 (ZP_01440547; SEQ ID NO: 4); 5: Chaetoceros neogracile KOPRI AnM0002 (as derived from EL620625, SEQ ID to NO: 5); 6: Truepera radiovictrix DSM 17093 (YP_003705905, SEQ ID NO: 6); 7: T. radiovictrix DSM 17093 (YP_003706581; SEQ ID NO: 7); 8: Gillisia limnaea R-8282 (ZP_09669334, SEQ ID NO: 8); 9: Krokinobacter (Dokdonia) sp. 4H-3-7-5 (YP_004429763, SEQ ID NO: 9). The underlined residues in SEQ ID NO: 1 appear to be conserved among NQ-rhodopsines and are, therefore, in one embodiment preferably not subject to any substitution.

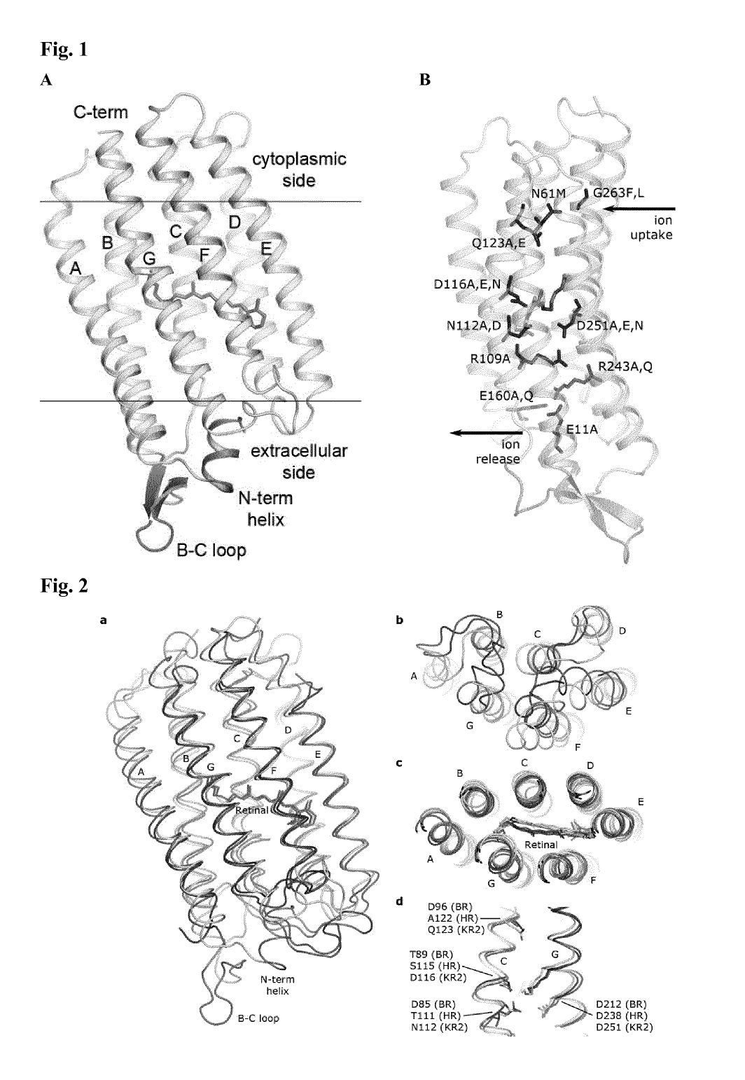

| SEQUENCES | |
|---|---|
| Krokinobacter eikastus KR2 (Uniprot ID N0DKS8; SEQ ID NO: 1) | |
| MTQELGNANF ENFIGATEGF SEIAYQFTSH ILTLGYAVML AGLLYFILTI KNVDKKFQMS | 60 |
| NILSAVVMVS AFLLLYAQAQ NWTSSFTFNE EVGRYFLDPS GDLFNNGYRY LNWLIDVPML | 120 |
| LFQILFVVSL TTSKFSSVRN QFWFSGAMMI ITGYIGQFYE VSNLTAFLVW GAISSAFFFH | 180 |
| ILWVMKKVIN EGKEGISPAG QKILSNIWIL FLISWTLYPG AYLMPYLTGV DGFLYSEDGV | 240 |
| MARQLVYTIA DVSSKVIYGV LLGNLAITLS KNKELVEANS | 280 |
| Citromicrobium sp. JLT1363 (ZP_08702831; SEQ ID NO: 2) | |
| MVAPITDNTV VSPPIPEVNT ALANQLGNIE NVVTLTMQQY TLGSLILMVS YGACFAFILF | 60 |
| FLMSVQNLAP RYRLVPILSA VVMASAGLSL LQEFSLWKDS YAFVDGLYRP LAENETFTNA | 120 |
| YRYGNWTITV PILLTQLAIA MGLRQGEIQR RSLRMGVPAV LMIWTGLYGQ FGEVGDFSHL | 180 |
| NLWGVVSSIF FLWLILEVRQ TLIAGISSTP DILKPWPNNL WWFFLATWGL YPIAYALPQL | 240 |
| GATAEIVIAR QGIYSLADIA SKLIYGIILA RFVLRRSAYE GYMPAAEALA SAPEKPNTGG | 300 |
| PGLRRAP | 307 |
| Citromicrobium bathyomarinum JL354 (ZP_06860850; SEQ ID NO: 3) | |
| MAAPIADPTV AEPQIAQADA AISAIENLVT LTSQQYHLGS LILMVSYAAF FAFILFFLMS | 60 |
| VQNLAPRYRL VPILSAVVMA SAGLTLLQEF GLWKESYAYV DGLYRPLAQN DSFSNAYRYG | 120 |
| NWTITVPILL AQLAIAMGLR QAEVHRRALR MAVPGVLMIW TGLYGQFGAV GDFSHLNLWG | 180 |
| VISSIFFLWL ILEVRQTLIA GLATTPDILK SWPSNLWWFF LASWGLYPLA YALPQLGATG | 240 |
| DLVVARQAIY SFADIASKLI YGIILARFVL RRSAFEGYMP AAEALAPTPD KPNTGGPGLR | 300 |
| RAP | 303 |
| Fulvimarina pelagi HTCC2506 (ZP_01440547; SEQ ID NO: 4) | |
| MQDTELFADQ LGNIENLVSL TVGQYTMGSL ILMVSYGAHF AFVLYFLMTS LQLAPRYRIV | 60 |
| PIMSAIVMVS AGLSLLREFN AWEQSYEFVE GMYQPLAENS TFTNAYRYGN WTITVPILLT | 120 |
| QLPLAFGLLR PELHVRAARM CIPALLMIWT GLVGQFGETG NYLRLNVWGV ISTIFFVWLI | 180 |
| IEVRGVISRA IAMGPAELAA WPKNIWWFFL AFWGLYPIAY ALPQLGHTGD IVVIRQLLYS | 240 |
| IADVFSKLVY GIILSRYVLR RSALEGYKPA IEALSRTPVD LSMSKNGGAG YGDGDR | 296 |
| Chaetoceros neogracile KOPRI AnM0002 (as derived from EL620625, SEQ ID NO: 5) | |
| MSAVLNVEGV RAACDRETGA AIANMENCLQ YSPLQFELVG HALVIGYAAQ AAGFIYFAMT | 60 |
| MNMTKGRNYQ LCSIYGMIVM LSAFLLLYNQ WAAWEDSFVL NAAGLYESGG VKLFSNGYRY | 120 |
| LNWSIDVPLL LLQLVLVSGL EVGTGFNKNL QVTTSGLLMI YLGYIGQFYE NPDSMVPLIA | 180 |
| FGVVGTVAYA IMLAIVLQCL SHAKKNFKTE SAKFKMNLVF WIFLIFWTIY PISYFMPVFS | 240 |
| YTAEGVVVRQ FIYTIADVVS KVIYCIILTQ ICMTTVMLLR EQIA | 284 |
| Truepera radiovictrix DSM 17093 (YP_003705905, SEQ ID NO: 6) | |
| MLTMENLLTY SPVSYSIVSN TLTLGYAAMA AGLVYFVTTS KRAAPPYRLS STLSAVVMVS | 60 |
| AFLALYQLHQ TWLSAFTFNG EVWEGGATAF NNGYRYINWS IDVPILLTQL LIVMGFTGAR | 120 |
| FRRLWLQFVV AGLAMIYTGY AGQFYEATDS ARLYLWGAIS TAFFLWILVL VRRTIFDPPD | 180 |
| ALPERAAGLM RGVWWVLLGS WLLYPGAYLM PVFALSEGGV VARQISFTVA DVVSKVIYGV | 240 |
| MLSRVAELRS QADGYAHALE SETQPNPDRR GGAGVAAVQP QGGRAPRKGR VHRS | 294 |
| T. radiovictrix DSM 17093 (YP_003706581; SEQ ID NO: 7) | |
| MQPSPHRLGN ATFENYVGLE SGYTEMAYQM SAHVLTVGYA IMLAALIYFI LTIRQVRPRY | 60 |
| RMSSILSVVV MVSAFLLLLI QQLNWTSALQ FDPATARYRL APEGVEGIVT AGDLFNNGYR | 120 |
| YLNWLIDVPM LLFQILFVVT LSRSSFASVR NQFWFSGVAM IITGYVGQFY EVTRPGLFFL | 180 |
| WGSISTVFFI HILIVMRRVI KEGVENAPDS AKGMLGAIWP LFLISWMLYP GAYLMPYLYS | 240 |
| FSLEPALSES AVVARHLTYT VADVTSKVIY GVLLAAAATR MSKAEGYDWE VQTA | 294 |
| Gillisia limnaea R-8282 (ZP_09669334, SEQ ID NO: 8) | |
| MFGDFNRVDN PILSLSYFKI QLTGIGLCRL LKPCLNFSKN IKHIKKFIKI NGNKFEIDYF | 60 |
| DMTQELGNAN FENFIGATEG FSEIAYQFTS HILTLGYAVM LAGLLYFILT IKKVDKKYQM | 120 |
| SNILSAVVMV SAFLLLYAQA ENWTTSFTFD ISRGKYFLEP NGDLFNNGYR YLNWLIDVPM | 180 |
| LLFQILFVVS LTKSKFSSIR NQFWFSGAMM IITGYIGQFY EVSNLTAFFV WGAISSVFFF | 240 |
| HILWVMKKVI NEGKEGLSAD AQKILSNIWV LFLVSWFLYP GAYLMPYLTG LDGFFFSEDG | 300 |
| VMARQLTYTI ADVCSKVIYG VLLGNLALKL SNNKEMVELS N | 341 |
| Krokinobacter (Dokdonia) sp. 4H-3-7-5(YP_004429763, SEQ ID NO: 9) | |
| MTQELGNANF ENFIGATEGF SEIAYQFTSH ILTLGYAVML AGLLYFILTI KNVDKKFQMS | 60 |
| NILSAVVMVS AFLLLYAQAQ NWTSSFTFNE EVGRYFLDPS GDLFNNGYRY LNWLIDVPML | 120 |
| LFQILFVVSL TTSKFSSVRN QFWFSGAMMI ITGYIGQFYE VSNLTAFLVW GAISSAFFFH | 180 |
| ILWVMKKVIN EGKEGISPAG QKILSNIWIL FLISWFLYPG AYLMPYLTGV DGFLYSEDGV | 240 |
| MARQLVYTIA DVCSKVIYGV LLGNLAITLS KNKELVEANS | 280 |
| C-terminal tag (SEQ ID NO: 10) | |
| LEHHHHHH | 8 |

### EXAMPLES

### Example 1

Expression Plasmid

Krokinobacter eikastus sodium pumping rhodopsin gene (Uniprot ID N0DKS8) coding DNA was synthesized de novo. The nucleotide sequence was optimized for E. coli expression using the GeneOptimizer™ software (Life Technologies, USA). The gene was introduced into the pSCodon1.2 expression vector (Staby™ Codon T7, Eurogentec, Belgium) via NdeI and XhoI restriction sites. Consequently, the expressed construct harbored an additional C-terminal tag with a sequence LEHHHHHH (SEQ ID NO: 10).

Protein Expression and Purification

E. coli cells of strain SE1 (Staby™ Codon T7, Eurogentec, Belgium) were transformed with the KR2 expression plasmid. Transformed cells were grown at 37° C. in shaking baffled flasks in an auto-inducing medium ZYP-5052 (Studier, F. W. Protein Expression and Purification 41, 207-234 (2005); incorporated herein by reference) containing 100 mg/L ampicillin. When glucose level in the growing bacterial culture dropped below 10 mg/L, 10 μM all-trans-retinal (Sigma-Aldrich, Germany) was added, the incubation temperature was reduced to 20° C. and incubation continued overnight. Collected cells were disrupted in M-110P Lab Homogenizer (Microfluidics, USA) at 25000 psi in a buffer containing 20 mM Tris-HCl pH 8.0, 5% glycerol, 0.5% Triton X-100 (Sigma-Aldrich, USA) and 50 mg/L DNase I (Sigma-Aldrich, USA). The membrane fraction of cell lysate was isolated by ultracentrifugation at 90000 g for 1 h at 4° C. The pellet was resuspended in a buffer containing 50 mM NaH₂PO₄/Na₂HPO₄ pH 8.0, 0.1 M NaCl and 1% DDM (Anatrace, Affymetrix, USA) and stirred overnight for solubilization. Insoluble fraction was removed by ultracentrifugation at 90000 g for 1 h at 4° C. The supernatant was loaded on Ni-NTA column (Qiagen, Germany) and KR2 was eluted in a buffer containing 50 mM NaH₂PO₄/Na₂HPO₄ pH 7.5, 0.1 M NaCl, 0.5 M imidazole and 0.1% DDM. The eluate was subjected to size-exclusion chromatography on 125 ml Superdex 200 PG column (GE Healthcare Life Sciences, USA) in a buffer containing 50 mM NaH₂PO₄/Na₂HPO₄ pH 7.5, 0.1 M NaCl, 0.05% DDM. Protein-containing fractions with the minimal A280/A525 absorbance ratio were pooled and concentrated, e.g. to 40 mg/ml.

Measurements of the Pump Activity in E. coil Cells

E. coli cells of strain C41(DE3) (Lucigen, USA) were transformed with the KR2 expression plasmid. Transformed cells were grown at 37° C. in shaking baffled flasks in an auto-inducing medium ZYP-5052 (Studier, F. W. Protein Expression and Purification 41, 207-234 (2005); incorporated herein by reference) containing 100 mg/L ampicillin and induced at the optical density OD₆₀₀ of 0.6-0.7 with 1 mM isopropyl β-D-1-thiogalactopyranoside (IPTG) and 10 μM all-trans-retinal. 3 hours after the induction the cells were collected by centrifugation at 3000 g for 10 min and washed three times with unbuffered salt solution (100 mM NaCl or KCl, 10 mM MgCl₂) with 30 min intervals between the washes to allow exchange of the ions inside the cells with the bulk. After that, the cells were resuspended in 100 mM NaCl or KCl solution respectively and adjusted to OD₆₀₀ of 8.5. The measurements were performed on three milliliters of stirred cell suspension kept at 1° C. The cells were illuminated for 5 minutes using the halogen lamp Intralux 5000-1 (VOLPI, Switzerland) and the light-induced pH changes were monitored with a pH meter LAB 850 (SCHOTT Instruments, Germany). Measurements were repeated under the same conditions after addition of 30 μM of CCCP and further addition of 20 mM of TPP⁺. For measurements at pH 5.6 the acidity was adjusted with 10 mM HCl and the cells were equilibrated for 30 minutes.

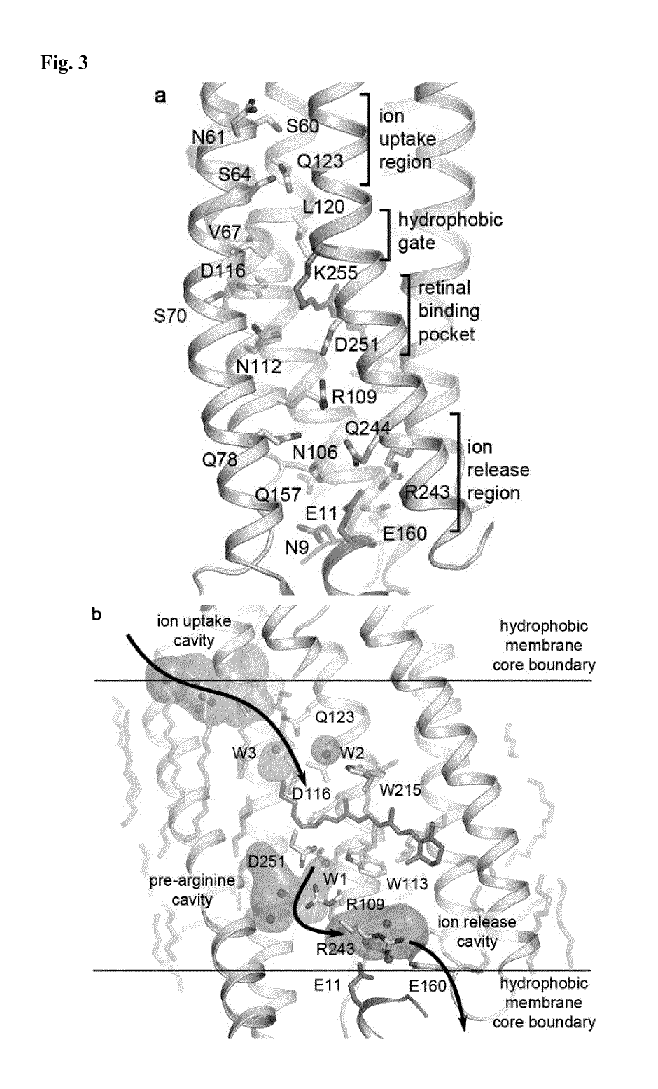

Upon dissolution of the crystal structure of KR2, an ion translocation pathway lined with polar and ionizable residues was hypothesized (FIG. 3a). There are three major cavities on this pathway (FIG. 3b). The first cavity protrudes from the protein surface towards Gln-123, situated at the position usually occupied by the proton donor residue in light-driven proton pumps (Lanyi, J. K. Annual Review of Physiology 66, 665-688 (2004); Bamann, C., et al. Biochimica et Biophysica Acta (BBA)-Bioenergetics 1837, 614-625 (2014)). Such wide opening of this cavity already in the ground state is unique among the microbial rhodopsins of known structure. The cavity is separated from the retinal by the hydrophobic residues Val-67 and Leu-120, conserved among bacterio- and proteorhodopsins. The second cavity precedes the Arg-109 side chain. The third cavity is situated in the region usually harboring the proton release group in light-driven proton pumps.

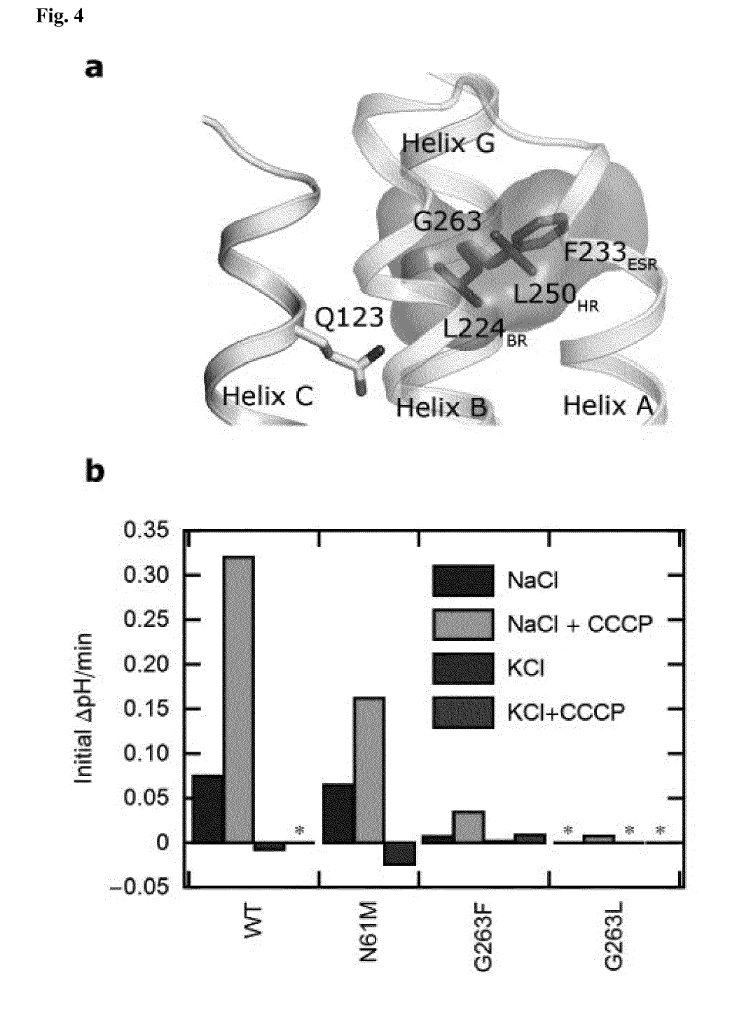

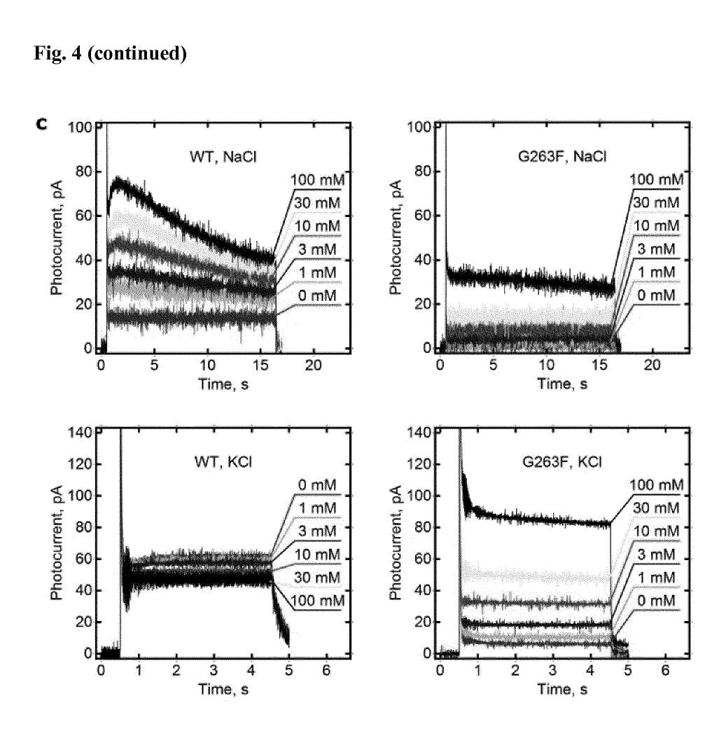

The cytoplasmic side of the protein is identical in all of the presented structures, and reveals a hydrophilic cavity protruding from the protein surface at the hydrophobic membrane core boundary towards the buried Gln-123, the residue replacing the proton donor residues of archaeal and bacterial proton pumps (FIG. 3b). Such protrusion is absent in other light-driven pumps, as there are much bulkier amino acids at the position of KR2's Gly-263 (FIG. 4a).

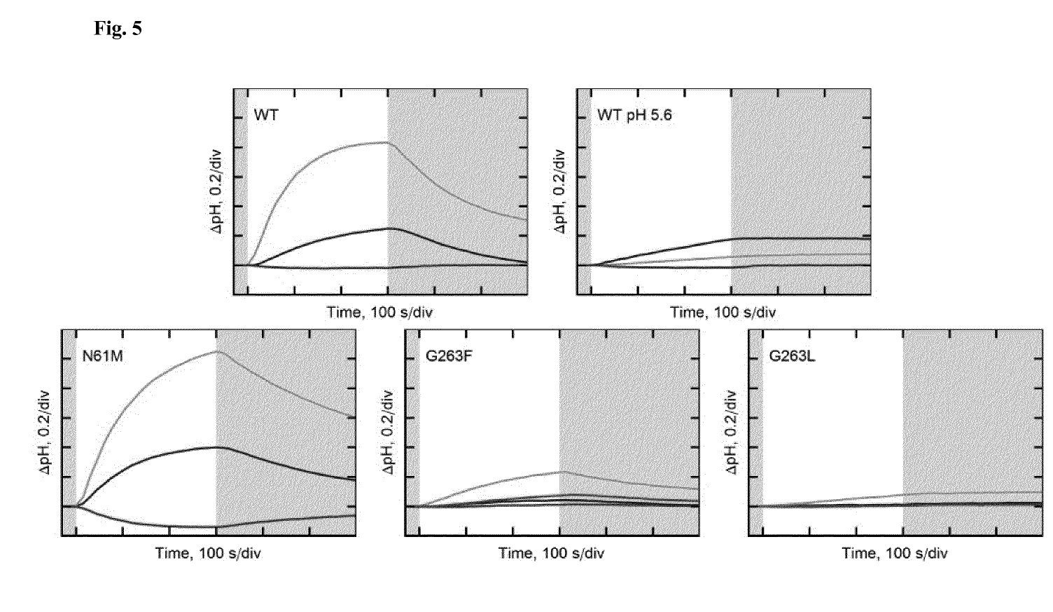

To determine the role of the cavity, we measured the activity of the mutants N61M, G263F and G263L (FIG. 4 and Fig.5). Mutation N61M, which makes the cavity less polar, results in lesser efficiency of sodium translocation and increased efficiency of proton translocation (FIG. 4b). Effects of the mutations of Gly-263 are more profound. While the sodium-pumping ability of the G263F mutant is severely impaired, the protein gained the potassium-pumping ability (FIG. 4b), as E. coli cells overexpressing KR2 alkalified the surrounding media under illumination in the KCI solution both in absence and presence of the protonophore carbonylcyanide-m-chlorophenylhydrazone (CCCP). This observation is supported by measurements of the photocurrents generated by KR2 reconstituted into liposomes.

Measurements of the Pump Activity in the Liposomes Attached to BLM

KR2 was reconstituted into liposomes (80% POPC, 10% POPG, 10% cholesterol (w/w), Avanti/Sigma Aldrich) at protein/lipid ratio of 7:100 (w/w). The detergent was removed by overnight stirring at 4° C. with the detergent-absorbing beads (Bio-Beads SM 2, BioRad). Optically BLMs were formed as described previously (Bamberg, E. et al. Biophys. Struct. Mechanism 5, 277-292 (1979); incorporated herein by reference). The electrolyte solution was 20 mM HEPES pH 7.4 without any Na⁺ or K⁺ ions. KR2-containing proteoliposomes were added to one of the compartments under gentle stirring. The system was illuminated with a mercury arc lamp (Osram HBO 100) at wavelengths >455 nm. Photosensivity of the samples developed over time and reached maximal current amplitudes after ˜30 minutes. 2 μM of the protonophore 1799 ((2,6-dihydroxy)-1,1,1,7,7,7-hexafluoro-2,6-bis(trifluoromethyl)-heptane-4-one) was added to both compartments, which effectively permeabilizes the compound membrane system for protons. Sodium and potassium titration was performed with NaCl and KCI solutions respectively by addition to both compartments of the cuvette. The system conductance remained constant during the titration. Subsequently, the membrane was made permeable for the cations, too, by the addition of the exchanger monensin. Photocurrents were measured under short-circuit conditions, so that no external driving force is generated. Further details of the system were as described (Bamberg, E. et al. Biophys. Struct. Mechanism 5, 277-292 (1979); incorporated herein by reference).

In the absence of Na⁺/K⁺ ions the wild type KR2 shows a stable stationary current that reflects the proton pumping activity of the protein. This pumping activity almost remains the same during potassium titration (FIG. 4c). However, the photocurrent amplitude rises significantly when the sodium concentration is raised (FIG. 4c). Declining curves at higher concentrations of sodium show that the protein is pumping sodium against the created chemical potential. This effect can be removed by the addition of the Na/H-exchanger monensin resulting in stable stationary currents.

The G263F mutant behaves differently. In the absence of Na⁺/K⁺ ions the stationary current is very low, showing that proton pumping is almost absent. Still the photocurrents preserve the dependence on sodium ions concentration (FIG. 4c), but the pumping became much less efficient. Addition of potassium ions results in drastic increase of the photocurrents (FIG. 4c), suggesting that G263F can pump potassium ions.

In contrast thereto, the G263L mutation resulted in the strongest inhibition of light-driven pumping. Based on the obtained data, we conclude that the ion uptake cavity plays a significant role in KR2 ion selectivity, and might be its selectivity filter.

### Example 2

The KR2 G263F K⁺-pumping mutant is inserted into cultivated hippocampal cells together with the light-gated cation channel channelrhodopsin 2(ChR2). Expression of the KR2 G263F mutant is expected to allow silencing these electrically excitable cells by light. A similar effect was demonstrated by Zhang et.al (Nature. 446(7136), 633-639 (2007)). In this publication the hyperpolarizing pump halorhodopsin (NphR) was used to inhibit the light induced firing of neurons, which were activated by the light induced depolarization via ChR2. The outwardly directed KR2 G263F K⁺-pump, however, is supposed to act in a much less invasive manner as NphR, because under physiological conditions the pump can use after light activation the naturally occurring potassium gradient. Therefore, KR2 G263F mimicks the action of endogenous outwardly directed K⁺ channels, which are responsible for the repolarization(silencing) of electrically excitable cells. These properties make KR2 G263F to an optimal optogenetic tool for silencing of neurons and muscle cells.

LIST OF REFERENCES

KR20130134792

KR20140011750

Kwon, S.-K. et al. Genomic Makeup of the Marine Flavobacterium Nonlabens (Donghaeana) dokdonensis and Identification of a Novel Class of Rhodopsins. Genome Biol Evol 5, 187-199 (2013).

Inoue, K. et al. A light-driven sodium ion pump in marine bacteria. Nat Commun 4, 1678 (2013).

Ernst, O. P. et al. Microbial and Animal Rhodopsins: Structures, Functions, and Molecular Mechanisms. Chem. Rev. 114, 126-163 (2014).

Grote, M., Engelhard, M. & Hegemann, P. Of ion pumps, sensors and channels—Perspectives on microbial rhodopsins between science and history. Biochimica et Biophysica Acta (BBA)-Bioenergetics 1837, 533-545 (2014).

Boyden, E. S., Zhang, F., Bamberg, E., Nagel, G. & Deisseroth, K. Millisecond-timescale, genetically targeted optical control of neural activity. Nat. Neurosci. 8, 1263-1268 (2005).

Oesterhelt, D. & Stoeckenius, W. Rhodopsin-like Protein from the Purple Membrane of Halobacterium halobium. Nature 233, 149-152 (1971).

Schobert, B. & Lanyi, J. K. Halorhodopsin is a light-driven chloride pump. J. Biol. Chem. 257, 10306-10313 (1982).

Lanyi, J. K. Bacteriorhodopsin. Annual Review of Physiology 66, 665-688 (2004).

Bamann, C., Bamberg, E., Wachtveitl, J. & Glaubitz, C. Proteorhodopsin. Biochimica et Biophysica Acta (BBA)-Bioenergetics 1837, 614-625 (2014).

Kolbe, M., Besir, H., Essen, L.-O. & Oesterhelt, D. Structure of the Light-Driven Chloride Pump Halorhodopsin at 1.8 Å Resolution. Science 288, 1390-1396 (2000).

Luecke, H., Schobert, B., Richter, H.-T., Cartailler, J.-P. & Lanyi, J. K. Structure of bacteriorhodopsin at 1.55 Å resolution. Journal of Molecular Biology 291, 899-911 (1999).

Gushchin, I. et al. Structural insights into the proton pumping by unusual proteorhodopsin from nonmarine bacteria. PNAS 110, 12631-12636 (2013).

Ho, B. & Gruswitz, F. HOLLOW: Generating Accurate Representations of Channel and Interior Surfaces in Molecular Structures. BMC Structural Biology 8, 49 (2008).

Studier, F. W. Protein production by auto-induction in high-density shaking cultures. Protein Expression and Purification 41, 207-234 (2005).

Bamberg, E. et al. Photocurrents generated by bacteriorhodopsin on planar bilayer membranes. Biophys. Struct. Mechanism 5, 277-292 (1979).

Deisseroth, K. Optogenetics. Nat. Methods 8, 26-29 (2011).

Chow et al. High Performance Genetically Targetable Optical Neural Silencing by Light-driven Proton Pumps. Nature 463, 98-102 (2010).

Santacruz-Toloza et al. Purification and Reconstitution of Functional Shaker K⁺ Channels Assayed with a Light-Driven Voltage-Control System. Biochemistry, 33(6), 1295-1299 (1994).

Zhang et al. Multimodal fast optical interrogation of neural circuitry. Nature. 446(7136), 633-639 (2007).

1.-15. (canceled)

16. A light-driven cation pump, wherein the light-driven cation pump comprises an amino acid sequence which has at least 70% sequence identity to the full length amino acid sequence of SEQ ID NO: 1 (Krokinobacter eikastus rhodopsin 2; KR2), and which comprises a substitution at a position corresponding to G263 in SEQ ID NO: 1, which substitution confers a potassium ion pumping ability.

17. The light-driven cation pump of claim 16, wherein the light-driven cation pump comprises, preferably consists of, the amino acid sequence of SEQ ID NO: 1 (KR2), except for a substitution at position G263 in SEQ ID NO: 1, which substitution confers a potassium ion pumping ability.

18. The light-driven cation pump of claim 16, wherein the substitution is selected from G263F and G263Y, preferably wherein the substitution is G263F.

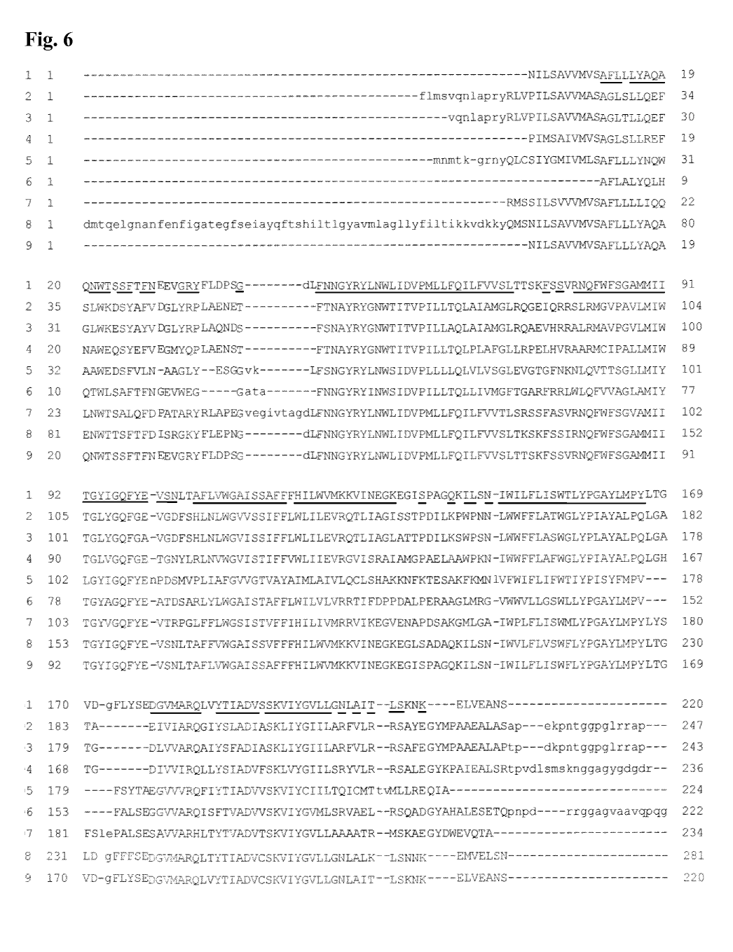

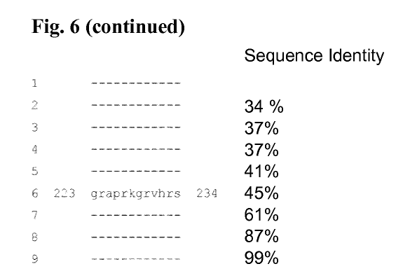

19. The light-driven cation pump of claim 16, wherein the light-driven cation pump does not comprise any substitution at a position in which the corresponding amino acid residue is identical among all NQ-rhodopsins in the alignment of FIG. 6.

20. The light-driven cation pump of claim 16, further comprising, in non-covalent linkage, a retinal or retinal derivative.

21. The light-driven cation pump of claim 20, wherein the retinal derivative is selected from the group consisting of 3,4-dehydroretinal, 13-ethylretinal, 9-dm-retinal, 3-hydroxyretinal, 4-hydroxyretinal, naphthylretinal; 3,7,11-trim ethyl -dodeca-2,4,6,8,10-pentaenal; 3,7-dimethyl-deca-2,4,6,8-tetraenal; 3,7-dimethyl-octa-2,4,6-trienal; and 6-7 rotation-blocked retinals, 8-9 rotation-blocked retinals, and 10-11 rotation-blocked retinals.

22. A nucleic acid construct, comprising a nucleotide sequence encoding the light-driven cation pump according to claim 16.

23. An expression vector, comprising a nucleotide sequence coding for the light-driven cation pump according to claim 16.

24. The expression vector of claim 23, wherein the vector is suitable for gene therapy.

25. The expression vector of claim 24, wherein the vector is suitable for virus-mediated gene transfer.

26. A recombinant host cell comprising the light-driven cation pump according to claim 16, the nucleic acid construct according to claim 22.

27. The recombinant host cell of claim 26, wherein the recombinant host cell is a mammalian cell which is
(i) a neuron; or
(ii) a muscle cell.

28. The recombinant host cell of claim 26, wherein the recombinant host cell is a mammalian cell which is
(i) a bipolar neuron, a pseudounipolar neuron, a multipolar neuron, a an anaxonic neuron, a basket cell, Betz cell, Lugaro cell, medium spiny neuron, Purkinje cell, pyramidal neuron, Renshaw cell, unipolar brush cell, granule cell, anterior horn cell, spindle cell, an afferent neuron, efferent neuron, or an interneuron, photoreceptor cell, rods and cones; or
(ii) a skeletal muscle cell, a smooth muscle cell, or a cardiac muscle cell.

29. A method of treatment, comprising the step of administering a light-driven cation pump according to claim 16.

30. A non-therapeutic method of actively transporting potassium ions across a membrane, comprising the step of introducing the light-driven cation pump according to claim 20 into said membrane.

31. The non-therapeutic method of claim 30, wherein said membrane is a biologic membrane.

32. The non-therapeutic method of claim 30, wherein the method is an in vitro method.

33. A method for silencing electrically excitable cells, comprising the step of introducing the light-driven cation pump according to claim 16 into an electrically excitable cell, and activating said light-driven cation pump, thereby silencing said electrically excitable cell.

34. The method of claim 33, wherein said method is an in vitro method.

35. An optogenetic research method, comprising the step of introducing the light-driven cation pump according to any claim 16 into a recombinant cell; or providing a recombinant host cell according to any claim 26, and activating said light-driven cation pump.

36. A method of high-throughput screening, providing the steps of providing a recombinant host cell according to claim 26, contacting the cell with a test substance, exciting the light-driven cation pump of the recombinant host cell, and identifying whether the test substance is an active agent which modulates the light-driven cation pump, a Ca⁺⁺-inducible potassium channel, or a BK channel of the recombinant host cell.
